(19) United States
(12) Patent Application Publication
(10) Pub. No.: US 2021/0315772 A1
(43) Pub. Date: Oct. 14, 2021

(54) System and method to remind acupuncturists to remove all needles from patients after treatments

(71) Applicant: Wellness Allied Inc, Natick, MA (US)

(72) Inventors: Weihui Li, Natick, MA (US); Tao ZHANG, Natick, MA (US)

(21) Appl. No.: 16/843,229

(22) Filed: Apr. 8, 2020

(51) Int. Cl. A61H 39/08; G16H 40/40; A61B 50/36

(52) U.S. Cl. CPC A61H 39/086; G16H 40/40; A61H 2201/5048; A61B 2560/045; A61B 2560/0462; A61B 50/362

(57) ABSTRACT

A system and method to remind acupuncturists to remove all needles from patients after acupuncture treatments. The system includes: a container for acupuncture needles, with at least three divided compartments: a first compartment for new acupuncture needle packages; a second compartment for used acupuncture needles; a third compartment for needle packaging materials; and an electronic weighing device to weigh the container with or without load. The method includes: loading enough new needle packages in the container; measuring the weight of the container and its load and displaying the total weight; automatic detection and warning of overload; automatic detecting whether the reset key has been pressed before acupuncture treatment; when the reset key is pressed, recording the current load value as the baseline weight and setting the relative reading to zero and displaying it on a screen; continuing to measure the weight of the container and its load; if the reading varies, determining whether the change of reading is caused by system drift or by the actual change of load; if the change of the readings is caused by system drift, automatically cancelling the drift value; displaying the difference between the measured weight and the reset baseline value on the display screen; based on the difference, reminding the acupuncturist to remove all the acupuncture needles from patients after the treatment before discharging the patient. The present invention solves the problem of leaving needles on the patient's body or on treatment tables, which often happens in the current practice of acupuncture and could lead to malpractice lawsuit.

### TECHNICAL FIELD

The present invention generally relates to acupuncture assistance techniques, and in particular, a system and method to remind acupuncturists to remove all the needles from patients after the treatments.

### BACKGROUND

Acupuncture has a history of thousands of years in China. Under the guidance of traditional Chinese medicine theory, acupuncture is done by inserting thin needles into specific body locations of patients at certain angles, followed by some stimulation techniques such as twisting and lifting to treat diseases.

Acupuncture is very effective in treating various disorders and has many advantages, including: 1) there is a wide range of indications for acupuncture treatment such as pains, anxiety, depression, gynecological problems, GI tract disorders; 2) the acupuncture treatment takes effect relatively rapid and significant. Through acupuncture stimulation, acupuncture can enhance the immune system, improve cardiovascular functions, manage pain and many more disorders; 3) acupuncture is relatively easy to implement; 4) the cost for acupuncture is relatively low compared to other modern medicine; 5) there are no or very few side effects. It is safe and reliable and can be combined with other therapies for synergic treatments.

These are the reasons why acupuncture is welcomed by the general population. Up to now, acupuncture has been used in more than 140 countries and regions around the world. It has been playing a big role in keeping people healthy.

With the possibility of cross-contamination between patients, majorities of the countries require single-use disposal acupuncture needles. FIG. 1 shows a sample of acupuncture needles with guiding tubes. The guiding tube helps the acupuncturist to insert the needle into the patients quickly by a tap to minimize pain and for better depth control of the needle. Most of the disposable needles have similar shapes and packaging. Some manufactures have multiple needles in one package. Some manufactures have plastic needle handles.

For the acupuncture treatments, the acupuncturists often forget to remove all the acupuncture needles after the acupuncture treatments. With acupuncture needles left in the patients, they could cause pain, or could get broken in the body and cause more severe harm to the patients. In some other situations, acupuncture needles could fall on treatment tables or get stuck in the table sheets or pillow. This will pose risks to next patients, the house cleaning persons or the acupuncturists themselves, if the needles stick into their skins. In extreme cases, if the previous patient carries infectious diseases, such as AIDS or hepatitis B, then next patients, the house cleaning persons or the acupuncturists themselves can be infected, and the consequence could be very severe. From the perspective of acupuncturists or acupuncture clinics, even if the accidents do not harm the patients, it will affect the patients' user experience, damage the brand of the acupuncturists and clinics, or lead to malpractice lawsuits.

### SUMMARY

According to the present invention, a system and method to remind acupuncturists to remove all needles from patients after the treatments are provided, comprising: a container for acupuncture needles, including at least three separate compartments: a first compartment for new acupuncture needle packages; a second compartment for used needles; a third compartment for packaging materials; and an electronic weighing machine to weigh the container and its load.

Optionally, one or more of the first compartment, the second compartment, and the third compartment include partitions.

Optionally, the container for the acupuncture needles is a cuboid.

Optionally, each compartment is a cuboid.

Optionally, the container for the acupuncture needles is a truncated cone with the small base on the bottom, or a cylinder.

Optionally, the second compartment for used acupuncture needles is at the center of the truncated cone or cylinder.

Optionally, the second compartment for used acupuncture needles is at the center of the truncated cone or cylinder, protruding upward above other compartments. The surroundings and the bottom are leak-proof. There is a lid at the top with an opening on the lid to put needles in. The opening has a locking mechanism for safety during the transition.

Optionally, the second compartment for the used acupuncture needles is integrated with other compartments, or it is a separate compartment. It is reusable, or it is a disposable compartment. When it is necessary to empty the second compartment, the needles are safely transferred to a sharps container, or the container is recycled directly with the used acupuncture needles. If the container is recycled directly, the container will be color-coded red and marked with biohazard and sharps signs.

Optionally, the second compartment for the used acupuncture needles is integrated with other parts.

Optionally, the second compartment for the used acupuncture needles are separate from the other parts of the container and is detachable.

Optionally, the second compartment for the used acupuncture needles is a disposable sub-container. When the second compartment needs to be emptied, the sub-container together with the used acupuncture needles are recycled. In this situation, the second compartment will be color-coded red and marked with biohazard and sharps signs

Optionally, the electronic weighing machine has a reset button, and the system is suitable for the following operations: before the acupuncture treatment, put the container on an electronic weighing machine, put new acupuncture needle packages in the first compartment of the container, and press the reset button to store the current weight as the reset baseline value and zero the relative weight reading; during the acupuncture treatment, the new acupuncture needle packages are taken away from the first compartment of the container, the packaging materials are putting back to the third compartment of the container and the electronic weighing machine displays negative readings for the relative weight; after the acupuncture treatment, the acupuncturist removes the needles from the patient and put them back to the second compartment of the container. If all the needles are put back to the container, the relative weight reading will be back to zero. Otherwise, the reading is still a negative value, indicating that some needles haven't been put back yet.

Optionally, before the acupuncture treatment, the container is placed on an electronic weighing machine, new acupuncture needle packages are placed in the first compartment, and a reset button is pressed. In response to the reset, the weight of the container and its load is recorded as the reset baseline weight and is set to zero and displays on the display unit. During the acupuncture treatment, new acupuncture needle packages are taken away from the container for acupuncture and the packing materials are put back the container. The electronic weighing machine continuously tracks the weight of the container and its load and calculates the difference between the current weight and the reset baseline value (is also called relative weight) and displays on the screen. During the treatment, the relative weight value is no longer zero but a negative value, showing that the current total weight is less than the reset baseline value and indicating that some needles have been put back to the container yet. In the whole process, the electronic weighing machine distinguishes the weight changes due to system drift and the weight change due to actual load change. If the weight change is due to the system drift, the system automatically cancels the drift value. During the treatment, if the acupuncturist finds that the acupuncture needles are not enough for the treatment, new acupuncture needle packages need to be added to the container. Before adding needles, the acupuncturist presses a “Save” key to save the relative weight (the weight of the needles that haven't been put back to the container yet). After the loading of new needle packages, the acupuncturist presses a “Load” key to load and display the previously saved relative weight and the acupuncturist continues the treatment. After the acupuncture treatment and all the used acupuncture needles are put back into the second compartment, the electronic compartment returns to reset zero status.

Optionally, the electronic weighing machine displays both the total weight of the container and its load and the relative weight comparing to the last reset baseline value.

Optionally, the electronic weighing machine reminds acupuncturists for proper operations based on the total weight and relative weight.

Optionally, the system includes an overload alerting unit, warning the acupuncturists when the total weight of the container and its load exceed the upper limit of the weighing machine's rating capacity.

Optionally, the system further includes automatic alerting system with LEDs of different colors, like red, yellow and green LEDs, to remind the acupuncturists for proper operations. One example of such operation: when an acupuncturist adds new acupuncture needle packages into the first compartment and the total weight of the container and its load exceed the rating capacity of the electric weighing machine, all the LEDs flash. Accordingly, the acupuncturist does one or more of the following operations to solve the problem: empty the second compartment for used needles; empty the third compartment for needle packaging materials; reduce the new acupuncture needle packages in the first compartment, until all the LEDs stop flashing.

Optionally, the system includes reset alerting unit, reminding the acupuncturist to reset the system if the relative weight reading is not zero before the acupuncture treatment.

Optionally, the reset alerting unit remind acupuncturists to reset the system before the acupuncture treatment. Before the treatment, if the relative weight value is positive, a yellow LED light is on; if the relative weight value is negative, a red LED light is on. Both LEDs remind the acupuncturist to press the reset button to zero the relative weight reading before treatments.

According to the present invention, a method to remind acupuncturists to remove all the needles from patients after treatment, including: weigh the total weight of the container and its load; automatically check if the system has been reset; after reset, record the present measured value as the reset baseline, set the relative weight value to zero and display it on the LCD screen; weigh the relative weight after the reset; the relative weight will be tracked continuously afterward and used to indicate if there are needles taken away or put back.

Optionally, the method further includes: after the treatment, the acupuncturist removes the needles from the patients and put them back to the second compartment of the container. The system will track if all the needles have been put back by weighing the relative weight. If not, a warning signal will be given.

According to another aspect of the present invention, a system and method to remind acupuncturist to remove all needles from patients after treatments further include the following operations: before the acupuncture treatment, placing new acupuncture needle packages in the first compartment, placing the container on the electronic weighing machine and pressing the reset key. During the acupuncture treatment, the new acupuncture needle packages are taken out of the first compartment of the container for acupuncture treatment. The packaging materials are put back to the third compartment of the container. At this moment, the electronic weighing machine displays a negative value, indicating that some needles haven't been put back to the container yet. After the acupuncture treatment, the acupuncturists remove needles from the patient and put back to the second compartment. When the electronic weighing machine has a relative weight reading back zero, it indicates that the acupuncturist has removed all the needles from the patients and put back all of them into the container. If the relative weight reading remains a negative value, the acupuncturist needs to keep looking for the missing needles, until the reading is zero. In the continuous measurement process, the relative weight changes either due to system drift or due to actual load changes. The algorithm of the system will differentiate whether the relative weight value change is due to the system drift or due to the actual load changes. If the relative weight data change is caused by system drift, the system automatically cancels the drift value.

According to another aspect of the present invention, a system to remind the acupuncturists to remove all the needles from patients after treatments include a container with at least three separate compartments: the first compartment for new acupuncture needle packages; the second compartment for used acupuncture needles; the third compartment for the leftover packaging materials after the needles are used; a weighing sensor to sense the weight of the container and its load; an A/D converter to convert the analog signal of the weight to digital signal; a microprocessor to receive the digital signal and process the signal; an LCD screen to display the data coming from the microprocessor; a user key system to receive user inputs, including a reset key being able to reset the relative weight value to zero, on- and off-key to turn on or off the electronic weighing machine, save and load key to save or load the current relative weight value; a memory storing computer-executable instructions, to receive, store, compare and display weight values and set off warning signals when appropriate.

The system and method to remind acupuncturists to remove all needles from patients after the treatment solve a current problem in the field of acupuncture, that is, in some cases, the acupuncturists often forget to remove all the needles from the patients and lead to medical accidents. The system and method have the following two advantages: 1) The system is simple and reliable and is easy to operate. Before the acupuncture, the acupuncturist puts new acupuncture needle packages into the system's container. If the relative weight is not zero, it will remind the acupuncturist to reset it. During the process of acupuncture, the acupuncturist takes the acupuncture needles away from the container for treatment, and then puts the left-over packaging materials back into the third compartment of the container. After the treatment, the acupuncture needles are removed from the patients and put back into the second compartment. At this time, the system will determine whether the acupuncturist has removed all the acupuncture needles from the patient by checking if the relative weight is brought back to zero. If the relative weight reading is brought back to zero, it indicates that the acupuncturist has put back all the needles that were used. Otherwise, there are still needles missing somewhere. The acupuncturist needs to search for them before discharging the patient. 2) Low cost. The components, like weight sensor, A/D convert, microprocessor are all mature technology and readily available at low price.

### BRIEF DESCRIPTION OF THE DRAWINGS

FIG. 1 shows an example of a commonly used acupuncture needle with guiding tubes.

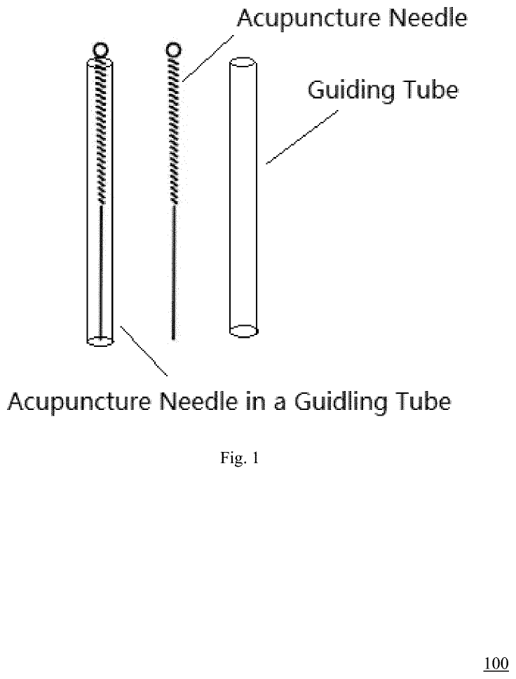

FIG. 2 shows an exemplary schematic structure of a system 100 to remind acupuncturists to remove all needles after treatment according to an embodiment of the present invention.

FIG. 3 shows an exemplary sample of a new acupuncture needle package.

FIG. 4 shows an exemplary sample of used acupuncture needles.

FIG. 5 shows an exemplary sample of left-over packaging materials.

FIG. 6 shows another exemplary schematic structure of a system 200 to remind acupuncturists to remove all needles after treatment according to an embodiment of the present invention, in which the container is truncated cone.

FIG. 7 shows a block diagram of a system 300 to remind acupuncturists to remove all the needles from patients after treatment according to an embodiment of the present invention.

FIG. 8 shows an exemplary flowchart of a method 400 to remind acupuncturists to remove all the needles after acupuncture treatment according to an embodiment of the present invention.

FIG. 9 shows an exemplary flowchart of a method 500 to remind acupuncturists to remove all needles from patients after treatment according to an embodiment of the present invention.

FIG. 10 shows an exemplary flowchart of an operation process 600 according to an embodiment of the present invention.

### DETAILED DESCRIPTION

Hereinafter, specific embodiments of the present invention will be described in detail with reference to the accompanying drawings.

Before the description, in order to facilitate the understanding of the invention, the basic idea of the present invention is outlined first. The basic principle of this patent is to use a specially designed container system for acupuncture needles and a high-precision electronic weighing system to weigh the container and its load. Acupuncturists take acupuncture needles out of the container for treatments, and then discard acupuncture needle packaging materials back to the container. After the treatment, acupuncturists collect all the needles used on patients and put them back to the container. Acupuncturists need to ensure that the weight displayed on the electronic weighing machine after the treatment is the same as the one before the acupuncture treatment. In general, in order to facilitate the acupuncturists to memorize, before the acupuncture treatment, the container with load is placed on the electronic weighing machine, and the electronic weighing machine is reset to zero by pressing the keys. After the acupuncture treatment, the acupuncturists only need to ensure that the electronic weighing machine's reading is back to zero. In this document, we do not make special restrictions on reset and tare to zero. Generally, it is expressed by reset. However, as mentioned earlier, the “zero” state is only for convenience to remember, and it is not a limitation. In fact, other values or labels are also feasible, such as displaying “reset”, “null”. In addition, due to system drift, if a nonzero number occurs during and after the treatment, as long as this value is significantly less than the weight of a single acupuncture needle, we believe that the system is also in a reset state. This document does not specifically limit the specific technical issues.

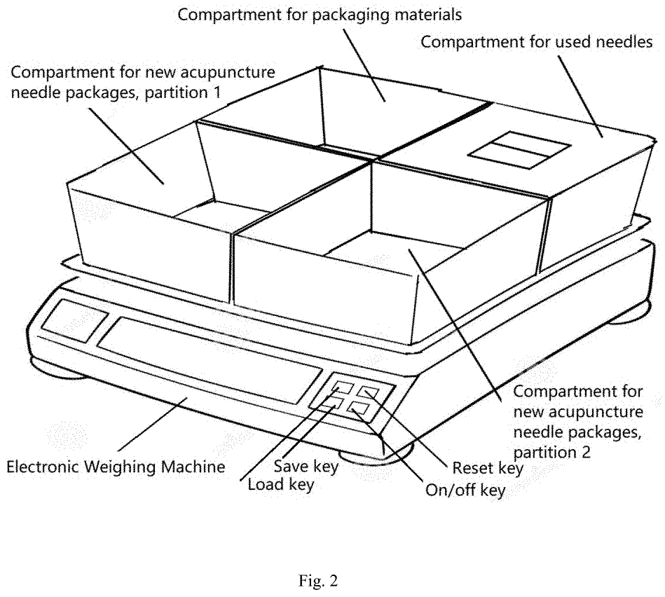

According to an embodiment of the present invention, a system and method to remind acupuncturists to remove all needles after treatment is provided. The system includes a container for acupuncture needles and an electronic weighing machine for weighing the container and its load. FIG. 2 shows an exemplary schematic structure diagram of a system 100 to remind acupuncturists to remove all needles from patients according to an embodiment of the present invention.

The container for acupuncture needles includes at least three separate compartments: a first compartment for new acupuncture needle packages; a second compartment for used acupuncture needles; a third compartment for left-over packaging materials.

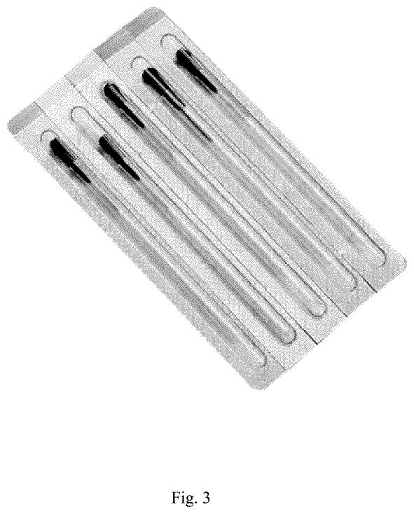

FIG. 3 shows an exemplary sample of a new acupuncture needle package.

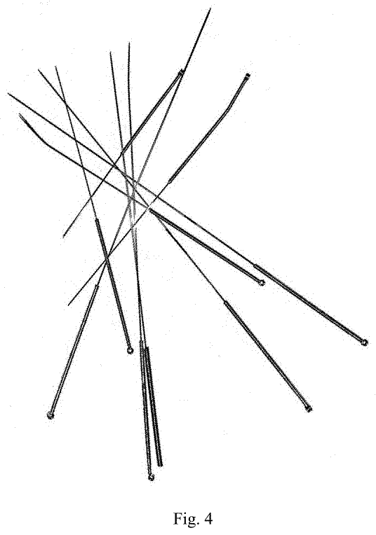

FIG. 4 shows an exemplary sample of used acupuncture needles.

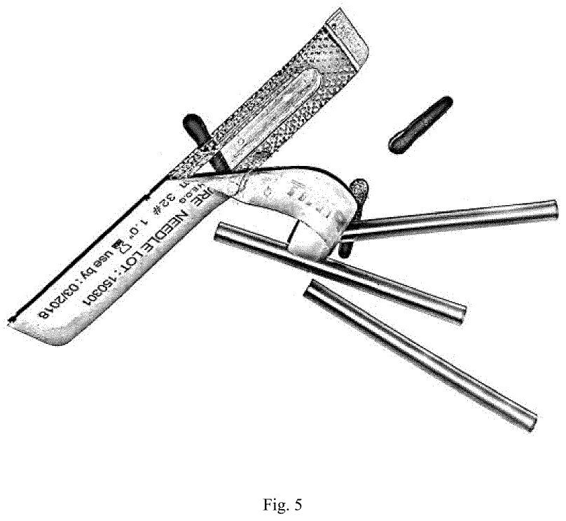

FIG. 5 shows an exemplary sample of left-over packaging materials after the needles have been taken away for treatment.

It should be noted that one or more of the first compartment, the second compartment, and the third compartment can include more partitions. In the example shown in FIG. 2, the first compartment for new acupuncture needle packages includes two separate partitions. This is an example and not a limitation, and one compartment can include more partitions. For example, the first compartment for new acupuncture needle packages includes three partitions or separate areas. In addition, the second compartment and/or the third compartment can also be divided into more partitions or separate areas. The separated areas can be adjacent or not adjacent. The number and locations of separate areas can be arranged as needed.

In the example shown in FIG. 2, the container for acupuncture needles has a cuboid shape.

In the example shown in FIG. 2, each compartment is cuboid.

In the example shown in FIG. 2, the second compartment for the used acupuncture needles has a lid at the top with an opening to put the used acupuncture needles in. This has the advantage of preventing acupuncturists from accidentally touching or reusing used acupuncture needles.

The shape of the container and the shape of each partition shown in FIG. 2 are taken as examples and not as a limitation, and other shapes can be adopted as needed.

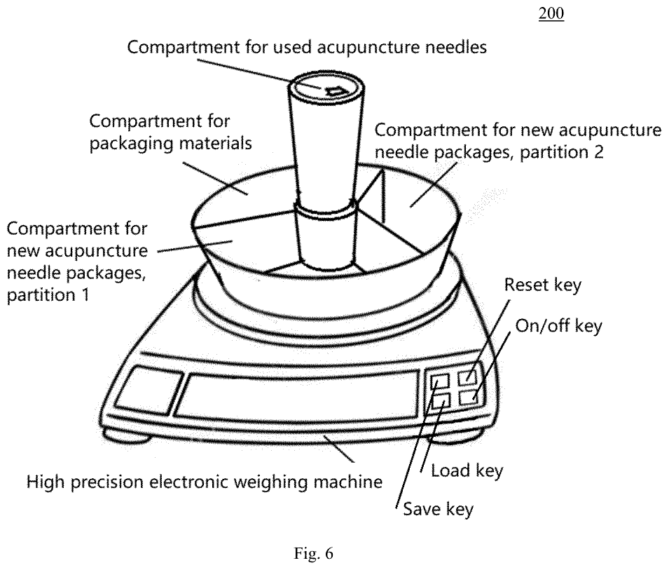

FIG. 6 shows another schematic diagram of a system structure 200 to remind acupuncturists to remove all needles from patients after the treatment, in which the container is in the shape of a truncated cone with a small base at the bottom.

In the example shown in FIG. 6, the second compartment for the used acupuncture needles is located at the center of the container and projects upwards above other parts and has a lid at the top. There is an opening on the lid to put the needles in.

In the example shown in FIG. 6, the first compartment for new acupuncture needle packages is divided into two partitions. Together with the third compartment for left-over packaging materials, they form a ring around the second compartment in the center.

The shape and compartment divisions of the container shown in FIGS. 2 and 6 are preferred examples, but not as limitations. Other container shapes and compartment division schemes can be designed as needed.

The second compartment for used acupuncture needles is integrated with other compartments, or is a separate component, or is reusable, or is a disposable container. When it is necessary to empty the used needles, the needles are safely transferred to a sharps container, or this sub-container is recycled directly together with the used acupuncture needles.

In one example, the second compartment for the used acupuncture needles is an integrated part with other parts.

In another example, the second compartment for the used acupuncture needles is a separate part from the other compartments and is removable.

In one example, the second compartment for the used acupuncture needles is a disposable sub-container. When it is necessary to recycle the used acupuncture needles, the sub-container is recycled together with the used acupuncture needles.

In another example, the second compartment for the used acupuncture needles is reusable, and when the second compartment for the used acupuncture needles needs to be emptied, the used acupuncture needles are directly transferred to a sharps container.

In one example, all or parts of the boundaries between the various compartments are adjustable.

In the specific implementation, the system needs to use a high-precision weight sensor to ensure that the system is able to detect a significant difference in the measured value when adding or removing a needle. Currently on the market, there are weight sensors already with a resolution of 0.001 grams, which fully meets the requirements. It should be noted that due to system drift, in the hardware and software design, factors related to system drift need to be considered. If the relative value after the reset is less than a threshold, we think the system is also in the reset state. This patent is not making limitation statement.

Preferably, the electronic weighing machine has a reset button, and the system is suitable for the following operations:

before the acupuncture treatment, new acupuncture needle packages are placed in the first compartment of the container; the container is put on an electronic weighing machine, and the relative weight value is reset to zero by pressing the reset key;

during the acupuncture treatment, new acupuncture needle packages are taken away from the first compartment of the container and the packaging material is put back to the third compartment of the container. The relative weight is not in reset zero state but negative values;

after the acupuncture treatment, all the acupuncture needles are removed from the patient and put back into the second compartment of the container. The electronic weighing machine returns to the reset state.

In one example, the system has an automatic alerting system to warn missing needles. Before the acupuncture treatment, reset key is pressed. The system receives and stores the current weight of the container and its load as the reset baseline value. During the treatment and after treatment, the electronic weighing machine keeps weighing the weight of the container and its load and compares the weight with the baseline weight. If the difference between the current weight and the reset baseline weight is not zero, the system will give a warning.

In addition, the weight sensor has a weighing range limit. In the system implementation, it is necessary to ensure that the total weight of the container and its load is less than the upper limit of the weight sensor's weighing range limit. It is hard for acupuncturists to determine if it is overloaded. The electronic weighing machine is needed to weigh the total weight to ensure that the total weight does not exceed the upper limit of the weighing machine's capacity.

In one example, the electronic weighing machine has an overload warning unit, giving an audible and/or visual warning when detects that the weight of the container and its load exceeds the weight limit of the weight sensor.

To achieve the above functions, the system simultaneously weighs and displays the total weight as well as the relative weight (the difference between the current weight and the reset baseline weight). For example, a key is set on the electronic weighing machine. Pressing the key will switch the display of total weight and relative weight alternatively. Another example, the electronic weighing machine is configured to display total weight and the relative weight at the same time.

In addition, according to an embodiment of the present invention, the system, such as an electronic weighing machine, is equipped with a misoperation warning unit. When the acupuncturist operates the device, sometimes the acupuncturist could make mistakes. For example, the acupuncturist might add new needle packages to the container while doing the acupuncture treatment without pressing the save and load keys. The change of weight usually is much larger than putting back left-over packaging materials during the treatment. In such a situation, the system will give a warning when detects such an abnormal change of weight.

In addition, according to an embodiment of the present invention, the system, such as an electronic weighing machine, is equipped with a reset operation warning unit. When the acupuncturist does not press the reset key before the treatment while the relative reading is nonzero, the system will have an audible and/or visual warning indicating that the system has not been reset and cleared. This prevents acupuncturists from forgetting to press the reset key before acupuncture treatment.

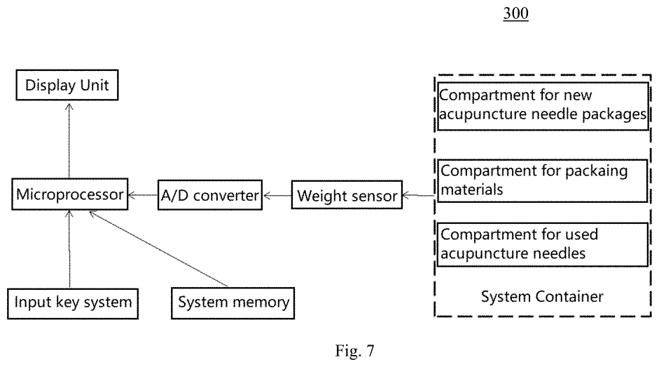

FIG. 7 shows a block diagram of the structure of a system 300 to remind acupuncturists to remove all needles after acupuncture treatment. Some or all of the parts indicated by these boxes can be integrated together. For example, the weight sensor, A/D converters, microprocessors, input keys, memory, and the display unit are integrated to build an electronic weighing machine. Other integration could be done, such as the integration of the display unit with the container.

As shown in FIG. 7, according to another embodiment of the present invention, a system and method to remind the acupuncturists to remove all the needles after the treatment, is provided, including:

A container for acupuncture needles, with at least three separate compartments: a first compartment for new acupuncture needle packages; a second compartment for used acupuncture needle; a third compartment for package materials with needles removed;

A weight sensor capable of sensing the weight of the container and its load;

An A/D converter that converts the weight value sensed by the weight sensor into a digital signal;

A microprocessor, receiving a digital signal from A/D converter, representing the measured weight;

A display unit that receives instructions from a microprocessor to display the weight data;

A user key system including the reset key, on/off keys, save/load keys. These keys receive user inputs to reset the relative weight reading, to turn on or off the electronic weighing machine, to save or load the data respectively.

A memory that stores computer-executable instructions. When the computer-executable instructions are executed by the microprocessor, they receive, store, compare, and display weight data.

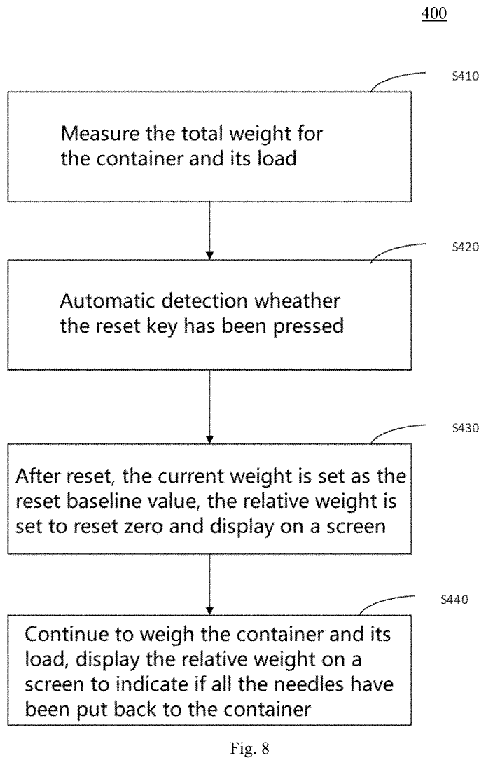

FIG. 8 shows an exemplary flowchart of a system and method 400 that remind acupuncturists to remove all the needles from patients after treatment.

In step S410, the weight of the container and the loaded contents is measured. The contents of the container generally include new acupuncture needle packages, used acupuncture needles, and packaging materials.

In step S420, it automatically detects whether the reset key is pressed. For example, the electronic weighing machine has a reset key, and when pressed, a reset signal is transmitted to the processor.

In step S430, when the reset key is pressed, the weight value measured at this time is used as the reset baseline value, and the relative weight value is set to the “reset clear value” and displayed on the display screen. Here, the reset clear value is preferably zero to facilitate memory, but other identifiers are also able to use as the reset clear value, such as “reset”, “null”.

In step S440, continue to measure the weight of the container and its load, and display the relative weight (the difference between the measured weight and the reset baseline value) on the display screen to indicate whether all needles used during the acupuncture treatment have been removed and put back to the container.

During acupuncture treatments, new acupuncture needle packages are continuously taken away for treatment, and the packaging materials are put back into the container. At those times, the relative weight is always less than the reset zero. Only when all acupuncture needles used during the treatment have been put back to the second compartment, the relative weight reading (the difference between the measured weight and the reset baseline value) will be less than the threshold value and displays the reset value on the display, for example zero. The acupuncturist then confirms that all the acupuncture needles have been removed from the patient, and there are no missing needles. On the contrary, if the absolute value of relative weight value is greater than the threshold, it indicates that some acupuncture needles have not been put back to the container yet. They are in the patient's body or have lost on the treatment table or floor. The acupuncturist further must look for the missing needles until all of them are recycled to the container.

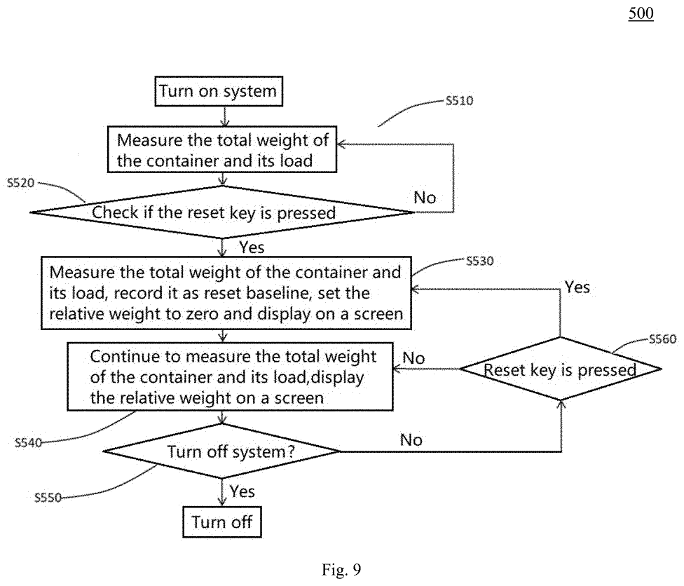

According to an embodiment of the present invention, FIG. 9 shows an exemplary flowchart of a system and method 500 to remind acupuncturists to remove all needles, in the situation of treating multiple patients in a row.

In step S510, the total weight of the container and its load (including new needle packages, used needles and left-over packaging materials) are measured. At some moments, it is possible that only new acupuncture needle packages are in the container while used needles and packaging materials are cleared away.

In step S520, the system monitors whether the reset key is pressed. If it is not pressed, return to step S510 to continue measuring the weight of the container and its load; if it is pressed, proceed to step S530.

In step S530, the total weight of the container and its load is measured. The measured weight value is recorded as the reset baseline value. The relative weight value (the difference between the current weight and the baseline value) is set to zero and displayed on the screen. As mentioned earlier, it is preferable to display the zero value as the reset clear value because it is easy to remember. However, this is not a limitation, and other values or identifications can be displayed, such as “Reset”, “NULL” or even “Initial Weight”.

In step S540, the total weight of the container and its load is continuously measured, and the difference between the current measured weight value and the reset baseline value, which is also called relative weight value, is displayed on the display screen.

Next, in step S550, the system monitors whether the power is turned off. Usually the acupuncturists or other operators turn off the power by pressing the shutdown button after confirming the removal of all the needles and no other patients to treat.

In step S550, if the power is not turned off, the process proceeds to step S560, monitoring whether the reset key is pressed. From time to time, after previous treatment and before the next treatment, the acupuncturists perform one or more of the following actions: clean out packaging materials, recycle used needles, add new acupuncture needle packages. Then the acupuncturist presses the reset button to start the next acupuncture operation.

In step S560, if the reset key is detected to be pressed, the process proceeds to step S530. Otherwise, the process proceeds to step S540.

In step S550, if it is confirmed that the shutdown signal is received, the system is turned off, and the entire process is ended.

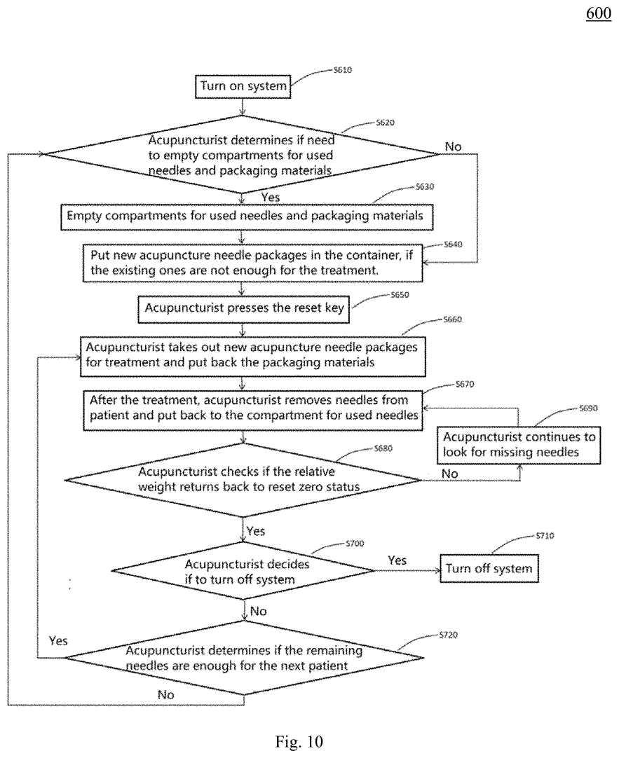

According to an embodiment of the present invention, FIG. 10 illustrates an exemplary operation diagram 600 for an acupuncturist.

In step S610, the acupuncturist turns on the system, e.g. turn on an electronic weighing machine by pressing on the on/off key.

In step S620, the acupuncturist determines whether it is necessary to clean up the compartments for used acupuncture needle and packaging materials.

If the acupuncturist determines in step S620 that it is necessary to clean up the compartments for used acupuncture needles and packaging materials, it proceeds to step S630. Otherwise it proceeds to step S640.

In step S630, the acupuncturist cleans up the compartments for used needles and/or packaging materials.

In step S640, the acupuncturist determines if there are enough new acupuncture needle packages in the container. If yes, proceeds to step S650. If no, places enough new acupuncture needle packages in the first compartment of the container and proceeds to step S650.

In step S650, the acupuncturist presses the reset key.

Step S660 is for the whole treatment process for a patient. During the treatment, acupuncturist repeatedly takes out acupuncture needles to treat the patient and puts the left-over packaging materials in the third compartment of the container.

At the end of the treatment, the acupuncturist proceeds to step S670 to remove all the needles from the patient and recycles all the acupuncture needles into the second compartment of the container.

The acupuncturist then proceeds to step S680 to check whether the relative weight reading is in a reset state of zero.

If the acupuncturist determines that the relative weight is not in the reset state in step S680, the process proceeds to step S690; otherwise, the acupuncturist determines that the relative weight reading is in the reset state, and proceeds to step S700.

In step S690, if the relative weight is not in a reset state, it indicates that some acupuncture needles have not been put back to the container yet, the acupuncturist continues to look for the used needles on the patient or on the treatment table or floor and put back to the container, and returns to step S680 to check whether the relative weight reading is in a reset state.

In step S700, the acupuncturist decides whether to turn off the system or continue to treat the next patient. If no more patient to treat, the acupuncturist proceeds to the step S710 and shuts off the system. Otherwise, the acupuncturist proceeds to step S720.

In step S710, the system is turned off and the acupuncturist finishes all the treatments.

In step S720, the acupuncturist checks if there are enough acupuncture needle packages for the next patient. If there are enough needle packages, the acupuncturist will proceed to step S660. Otherwise, the acupuncturist proceeds to step S620.

The system and method to remind acupuncturists to remove all needles after acupuncture treatment solve a current problem in the field of acupuncture, that is, in some cases, the acupuncturist forgets to remove all the needles used on patients and cause adverse effects on patients or other related staff. The system has the following characteristics: 1) Simple equipment, reliable performance and easy to operate. Before the acupuncture treatment, the acupuncturist puts the new acupuncture needle packages into the device container and reset the system. During an acupuncture treatment, the acupuncturist takes the acupuncture needles from the container and put back the packaging materials to the third compartment of the container. After the treatment, the acupuncturist removes the used acupuncture needles from the patients and put them back into the second compartment of the container. The acupuncturist only needs to verify that the electronic weighing machine's relative weight returns to the reset state before discharging the patient. 2) Low cost. The components used in the system, e.g. weight sensor, the A/D converter, the memory, the microprocessor, are mature products at low cost.

The embodiments of the present invention described above are exemplary, not exhaustive. They are also not limited to the disclosed embodiments. Many modifications and variations will be apparent to those skilled persons in the field without departing from the scope and spirit of the embodiments described. Therefore, the protection scope of the present invention shall be subject to the protection scope of the claims.

1. A system to remind an acupuncturist to remove all needles from patients after acupuncture treatments, including:
a container for acupuncture needles, including at least three separate compartments: a first compartment for new acupuncture needle packages; a second compartment for used acupuncture needles; a third compartment for packaging materials;
an electronic weighing device to weigh the container and its load.

2. The system of claim 1, wherein one or more of the first compartment, the second compartment, and the third compartment includes partitions.

3. The system of claim 1, wherein the container for acupuncture needles is cuboid.

4. The system of claim 3, wherein each compartment is a cuboid.

5. The system of claim 1, wherein the container for acupuncture needles is a truncated cone with a smaller base on the bottom or is a cylinder.

6. The system of claim 5, wherein the second compartment for the used acupuncture needles is at the center of the container.

7. The system of claim 6, wherein the second compartment for the used acupuncture needles is at the center of the container and protrudes upward from the rest of other compartments, with the surroundings and bottom being leak-proof, the top having a lid and an opening on the lid with locking mechanism to put in used needles.

8. The system of claim 1, wherein the second compartment for the used acupuncture needles is an integrative part of the whole container

9. The system of claim 1, wherein the second compartment for the used acupuncture needles is a separate part from other compartments and is removable.

10. The system of claim 1, wherein the second compartment for the used acupuncture needles is a disposable container of color-coded red, with leak-proof surrounding and bottom, having “biohazard”, “sharps” symbols and text on the sides, a lid on the top and an opening with locking mechanism on the lid to put used needles in, and being recycled with the used needles when the compartment is full.

11. The system of claim 1, wherein the second compartment for the used acupuncture needles is a reusable container with used needles being directly transferred to a sharps container when the second compartment needs to be emptied.

12. The system of claim 1, wherein all or part of the boundaries between different compartments are adjustable.

13. The system of claim 1, wherein the electronic weighing machine comprises a display screen, LED lights, on/off key, reset key, power switch, save key, load key, weighing top for the container, microprocessor, power cord, rechargeable battery unit.

14. The system of claim 13, wherein the electronic weighing machine has a reset button, is able to track the total weight and the relative weight after the reset, and the system is adaptable for the following operations:
before acupuncture treatment, the container is placed on the electronic weighing machine; new acupuncture needle packages are placed in the first compartment; and the reset button is pressed to record the present weight as the reset baseline value, set the present relative weight to zero and display the value on the screen;
during the acupuncture treatment, the new acupuncture needle packages are removed from the first compartment of the container; and packaging materials are put back to the third compartment of the container; the electronic weighing machine continuously weighs the container and its load and calculate the relative weight between current weight and the reset baseline weight and display the value on the screen; a negative relative weight indicates that present load is less than the reset baseline weight and there are needles taken away from the container and haven't been put back;
during the acupuncture treatment, the electronic weighing machine keeps weighing the container and its load and at the same time determines if reading change being due to system drift or actual change of the load; and automatically cancels the system drift if it is due to the system drift;
during the acupuncture treatment, if the “save” key is pressed, the system saves the current relative weight; and if the “load” key is pressed, the system reloads the saved relative weight and display it on the screen;
when the acupuncture treatment is done, all the needles are removed from patients and put back to the second compartment of the container until the electronic weighing machine has the relative weight being back to zero again.

15. The system of claim 1, wherein the electronic weighing machine is operable to record and display the total weight as well as the relative weight between the present weight and the reset baseline weight.

16. The system of claim 1, wherein the electronic weighing machine further comprise automatic alerting system that reminds the acupuncturist to perform correct operations based on the total weight value and relative weight value, through LED of different colors:
before the acupuncture treatment, the alerting system reminds the acupuncturist to press the reset key through a yellow LED if the relative weight reading is not zero;
after the reset key is pressure and the relative weight reading is zero, a green LED tells the acupuncturist to start the treatment;
during the acupuncture treatment, with the acupuncture needles being taken away and the relative weight reading being negative, a red LED reminds the acupuncturist that some needles haven't been put back yet;
after the acupuncture treatment, with all the acupuncture needles being put back to the container, a green LED light tells the acupuncturist that it is safe to discharge the patient;
any time before, during or after the acupuncture treatment, if the acupuncturist adds new acupuncture needle packages to the container and if the total weight is above the upper limit of the weight sensor, all LEDs flashes to warn the acupuncturist to empty the second compartment for used acupuncture needles, or empty the third compartment for packaging materials, or reduce the new acupuncture needle packages in the first compartment.

17. A method performed with a system to remind acupuncturists to remove all needles from patients after treatments,
the system includes:
a container for acupuncture needles, including at least three separate compartments: a first compartment for new acupuncture needle packages; a second compartment for used acupuncture needles; a third compartment for packaging materials;
an electronic weighing machine with a reset button and a display screen to weigh the container and its load;
and the method includes:
before the acupuncture treatment, the acupuncturist adds new acupuncture needles to the first compartment of the container, put the container on an electronic weighing machine, and press the reset button;
during the acupuncture treatment, with the new acupuncture needle packages being taken away from the first compartment of the container, and the packaging materials being put back to the third compartment of the container, the electronic weighing machine has a negative relative weight reading, indicating that some needles haven't been put back yet;
after the acupuncture treatment, the acupuncturist removes the needles from the patient and put back to the second compartment of the container and the relative weight reading will be back to zero when all the needles are put back to the container or the acupuncturist needs to continue to look for missing needles if the relative weight remaining a negative value.

18. A system to remind acupuncturists to remove all needles from patients after treatments, including:
a container for acupuncture needles, including at least three separate compartments: a first compartment for new acupuncture needle packages; a second compartment for used acupuncture needles; a third compartment for packaging materials;
a weight sensor, capable of measuring the total weight of the container and its load and tracking the changing of the weight;
an A/D converter that converts the weight sensed by the weight sensor into a digital signal;
a microprocessor, receiving a digital signal of the weight converted by the A/D converter;
a display unit that displays weight values coming from the microprocessor;
a keypad system receiving the user's inputs, including on and off keys, a reset key, save and load keys to save and load data;
a memory storing computer-executable instructions to receive, store, compare, display weight values and set off alerting signals when instructions are executed by the microprocessor.
